SEQUENCE LISTING (1 sequence)

SEQ ID NO: 1 - DNA, 13 bp, Artificial Sequence
  Example of DNA sequence
agtcatcgtagct

(19) United States
(12) Patent Application Publication
(10) Pub. No.: US 2014/0179909 A1
(43) Pub. Date: Jun. 26, 2014

(54) MICROFLUIDIC DEVICE FOR NUCLEIC ACID EXTRACTION AND FRACTIONATION

(71) Applicants: Jonathan James O'Halloran, Uckfield (GB); Elaine Harrington Warburton, Purley (GB); Matthew Daniel Solomon, Hughesdale, VIC (AU); John Edward McCormack, North Warrandyte, VIC (AU); Matthias Schuenemann, Brunswick, VIC (AU); David James Briggs, Lancefield, VIC (AU); Mindy Lee Andre, Croydon, VIC (AU)

(72) Inventors: Jonathan James O'Halloran, Uckfield (GB); Elaine Harrington Warburton, Purley (GB); Matthew Daniel Solomon, Hughesdale, VIC (AU); John Edward McCormack, North Warrandyte, VIC (AU); Matthias Schuenemann, Brunswick, VIC (AU); David James Briggs, Lancefield, VIC (AU); Mindy Lee Andre, Croydon, VIC (AU)

(73) Assignee: QUANTUMDX GROUP LIMITED, PURLEY, SURREY (GB)

(21) Appl. No.: 13/990,732

(22) PCT Filed: Nov. 30, 2011
(86) PCT No.: PCT/IB2011/003016

§ 371 Date: Dec. 18, 2013

Related U.S. Application Data
(60) Provisional application No. 61/418,305, filed on Nov. 30, 2010.

(51) Int. Cl. C12N 15/10; B32B 37/12

(52) U.S. Cl. CPC C12N 15/1017; B32B 37/12
USPC 536/25.41; 422/534; 156/281

(57) ABSTRACT

Various embodiments of the present disclosure generally relate to molecular biological protocols, equipment and reagents for the extraction and fractionation of DNA molecules, from whole or lysed samples, in a single flow-through device.

### CROSS-REFERENCE TO RELATED APPLICATIONS

This application claims the benefit of priority under 35 U.S.C. §119(e)(1) to U.S. Provisional Application No. 61/418,305 filed on Nov. 30, 2010.

### BACKGROUND OF THE INVENTION

1. Field of the Invention

Various embodiments of the present disclosure generally relate to molecular biological protocols, equipment and reagents for the extraction and fractionation of DNA molecules, from whole or lysed samples, in a single flow-through device.

2. Description of the Related Art

DNA is a long polymer consisting of units called nucleotides. The DNA polymers are long chains of single units, which together form molecules called nucleic acids. Nucleotides can be one of four subunits (adenine (A), cytosine (C), guanine (G) & thymine (T)) and, when in a polymer, they may carry the genetic information in the cell. DNA comprises two long chains of nucleotides comprising the four different nucleotides bases (e.g. AGTCATCGTAGCT . . . etc) with a backbone of sugars and phosphate groups joined by ester bonds, twisted into a double helix and joined by hydrogen bonds between the complementary nucleotides (A hydrogen bonds to T and C to G in the opposite strand). The sequence of nucleotide bases along the backbone may determine individual hereditary characteristics, or other acquired diseases, such as cancer.

The central dogma of molecular biology generally describes the normal flow of biological information: DNA can be replicated to DNA, the genetic information in DNA can be ‘transcribed’ into mRNA, and proteins can be translated from the information in mRNA, in a process called translation, in which protein subunits (amino acids) are brought close enough to bond, in order (as dictated by the sequence of the mRNA & therefore the DNA) by the binding of tRNA (each tRNA carries a specific amino acid dependant on its sequence) to the mRNA.

To study, or analyze the sequence and biology of DNA or RNA from a sample it is usually necessary to extract, or isolate, the nucleic acids from the rest of the clinical or biological sample (i.e. other cellular components such as lipids, carbohydrates, proteins, etc.). This is presently performed by a number of methods by those familiar with the art. These methods are described briefly below.

The standard methodology consists of a protocol with different variations depending upon application and sample type, begins with cell disruption or cell lysis, to release the DNA. This is commonly achieved by mechanical lysis (such as grinding, or grinding tissue in liquid nitrogen), sonicating, enymatically or chemically (such as adding a chaotropic salts (e.g. guanidinium thiocyanate) to the sample). The cells lipid membranes and other lipids, are usually removed by adding a detergent and the proteins usually removed by adding a protease (such as Protinase K, optional but almost always done). Water-saturated phenol, chloroform allows for phase separation by centrifugation of a mix of the aqueous sample and a solution, containing resulting in an upper aqueous phase and a lower organic phase (mainly chloroform). Nucleic acid is found in the aqueous phase, while proteins are found in organic phase. In a last step, RNA is recovered from the aqueous phase by precipitation with ice cold 2-propanol or ethanol. DNA will be located in the aqueous phase in the absence of guanidinium thiocyanate. Since DNA is insoluble in these alcohols, it will precipitate and aggregate, giving a pellet upon centrifugation. This step also removes alcohol-soluble salt. Adding a chelating agent to sequester divalent cations such as Mg2+ and Ca2+ prevents dnase enzymes from degrading the DNA. Cellular and histone proteins bound to the DNA can be removed either by adding a protease or by having precipitated the proteins with sodium or ammonium acetate, or extracted them with a phenol-chloroform mixture prior to the DNA-precipitation.

A second method isolates DNA from a lysate (regardless of what method of lysis is used) by virtue of its ability to bind to silica in the presence of high concentrations of chaotropic salts (Chen and Thomas, 1980; Marko et al. 1982; Boom et al. 1990). The DNA can bind to any silica surface, whether this is pillars with microfluidics cassettes, silica coated paramagnetic beads, a silica filter within a spin column, or other silica surface. The chaotropic salts are then removed with an alcohol-based wash and the DNA eluted in a low-ionic-strength solution such as TE buffer (a buffer consisting of tris hydroxymethylaminomethane (‘Tris’) and Ethylenediaminetetraacetic acid (‘EDTA’)) or water. DNA binds to silica because of dehydration and hydrogen bond formation, which competes against weak electrostatic repulsion (Melzak et al. 1996). Hence, a high concentration of salt will help drive DNA adsorption onto silica, and a low concentration will release the DNA. As the DNA is bound to the silica surface the rest of the cellular and other debris is simply washed away with wash buffers prior to eluting the DNA bound to the silica in either H₂O or TE buffer.

The ChargeSwitch® (Invitrogen) methodology sees negatively charged DNA (through their negatively charged phosphate backbone) in a lysate bind to a special ligand that acquires a positive charge at low pH values (<6.5). Proteins and other impurities removed from the ChargeSwitch-bound nucleic acids through the use of aqueous wash buffers. Nucleic acids can then be released from the ChargeSwitch® ligand when the pH of the surrounding media is raised (>8.5) and the positive charge is neutralized.

The Nexttec DNA isolation system, allows purifying DNA with a single centrifugation step within four minutes following cell lysis. It is up to five times faster than currently used DNA isolation systems. This is possible through a proprietary sorbent matrix, which, in a reversal of silica based methods, retains inhibiting substances, such as proteins and low molecular weight substances and lets pass the pure DNA within a lysed sample. One limitation of this method is that it relies on a long enzymatic lysis step, at 60° C.

The methodologies presently used by those skilled in the art are deployed in tubes, spin-columns or plates and require substantial hands on operator time and multiple steps and thus present a bottle-neck for the analysis of DNA. The use of liquid handling machines and deployment of the technologies presented above in multi-well plates, utilizing vacuums to draw the samples and reaction solutions in to the active matrix for each technology, has been used for higher through put use, however these require batching of samples to make them cost effective and still cause a bottle-neck. For many applications, such as molecular analysis of clinically relevant DNA at the point of care, which is fast becoming recognized as the only methodology to control the emerging drug resistance problems in infectious diseases, especially in developing and third world nations, these methodologies are unsuitable and can not be readily deployed in point of care devices.

Some methodologies, such as coating micro-pillars or other features and/or structures in microfluidic channels, or paramagnetic beads with silica surfaces have been translated and deployed in microfluidics devices, however the requirement for multiple wash steps and buffers means the fluidic programming and the microfluidic device design itself is complex, thus making the devices expensive.

### SUMMARY OF THE INVENTION

In one embodiment a device is disclosed for simultaneously extracting and fractionating DNA from a lysate or a whole sample, the device comprising a single flow-through microfluidic channel, the channel comprising buffer and reagent chambers and a sorbent filter.

The sorbent filter may comprise a support at least partially covered by a polymeric coating comprising polyaniline or derivatives thereof.

The device may comprise a glass material.

The device may comprise a PDMS material.

In an embodiment of the device, the single flow-through microfluidic channel comprises a bulbous structure at an inlet thereof and wherein the bulbous structure tapers to a thinner structure at an outlet thereof.

In some embodiments, the sorbent filter comprises a series of solid or hollow microstructures fabricated into the walls of the microfluidc channel.

In some embodiments, the sorbent filter is lose and packed within the microfluidic channel

In another embodiment, the sorbent filter is a matrix configured to bind cellular and other clinical/biological sample material other than nucleic acids.

A method is disclosed in accordance with some embodiment of the invention for fabricating a microfluidics device to extract and fractionate DNA from a sample. The method comprises: forming at least two blank layers having a channel; forming an adhesive layer with ports cut through the layer corresponding to an inlet and an outlet port of the channel; vacuum packing a portion of the channel with a sorbent material; and aligning the blank layers with the adhesive layer therebetween and bonding the layers together under pressure.

A method for simultaneously extracting and fractionating DNA from a lysate or a whole sample is disclosed in another embodiment. The method comprises: providing a device comprising a single flow-through microfluidic channel, the channel comprising buffer and reagent chambers and a sorbent filter; applying the sample to an inlet of the single flow-through microfluidic channel; activating the sorbent filter with a buffer; flowing the sample into the portion of the channel containing the sorbent filter; flowing the sample through the channel and looping it though the portion of the channel containing the sorbent filter from between one and ten times; and flowing the extracted and fractionated DNA out of the device.

In one variation, the sample is flowed into the portion of the channel containing the sorbent filter and then incubated therein for between 15 seconds and 15 minutes, before being flowed out the device. In another variation, the sample is flowed into the portion of the channel containing the sorbent filter and then oscillated back and forth within the sorbent filter channel, before flowing the sample out of the device.

In some embodiments, the sorbent filter is activated with a buffer, and lysed sample is flowed into the channel containing the filter and the sample flowed through the channel to the end, resulting in a pure, or near pure DNA solution.

The resultant eluate is sufficiently pure and concentrated to be detected in an agarose gel and can be used in PCR, RT-PCR, DNA sequencing, hybridization experiments and can be detected in nanobiosensors such as nanopores, carbon nanotubes and nanowire biosensors.

In some embodiments, the buffer(s) and other reagents are stored off cassette and delivered via the fluidics of an external device.

### BRIEF DESCRIPTION OF THE DRAWINGS

FIG. 1 depicts an exploded schematic view of a microfluidics device for DNA extraction and fractionation.

FIG. 2 depicts a top view of a microfluidic device.

FIG. 3 depicts a bottom view of a microfluidic device.

FIG. 4 illustrates a polycarbonate insert of a microfluidic device.

FIG. 5 illustrates a polycarbonate shell of a microfluidic device.

FIG. 6 illustrates a laser-cut double-sided tape layer which may be used to bond the insert (FIG. 4) and shell (FIG. 5) together, thereby forming the microfluidic channels of the microfluidic device.

FIG. 7 illustrates a shell and insert laminated to cap fluid reservoirs.

FIG. 8 depicts the filters placed into the cartridge insert at the inlet and outlet of the sorbent packed chamber to ensure that the sorbent material remains in place.

FIG. 9 shows a double-sided tape layer being applied to the insert to hold the cassette halves (insert and shell) together and to create the microfluidics channels.

FIG. 10 illustrates a sorbent chamber filled under vacuum.

FIG. 11 illustrates the top view of an assembled nucleic acid extraction microfluidics device.

FIG. 12 illustrates the bottom view of an assembled nucleic acid extraction microfluidics device.

FIG. 13 shows the results from running 80 μl of 11.0 μg/ml salmon sperm through an extraction experiment using the extraction cassette and bench marking it with Nexttec clean column DNA extraction.

FIG. 14 shows PCR from eluate fractions from lysed human blood passed through the extraction microfluidics device. FIG. 14a shows the mass ladder in lane 1 and eluates 1 through 11 in lanes 2 to 12. FIG. 14b shows a mass ladder in lane 1 with lanes 2-10 containing eluate fractions 13 to 21.

FIG. 15 illustrates a gel image from a BioAnalyzer analysis of eluate fractions that separate DNA based upon size.

FIG. 16 shows an alternative embodiment of a DNA extraction and fractionating device. A.

FIG. 17 is a perspective view showing a point of care device and microfluidics cassette that includes the DNA extraction and fractionation device within the cassette.

FIG. 18 is a schematic view showing the components of a microfluidics cassette designed for handheld diagnostics in accordance with some embodiments.

FIG. 19 illustrates a microfluidics cassette design designed for handheld sequencing in some embodiments.

### DETAILED DESCRIPTION OF THE PREFERRED EMBODIMENT

In some embodiments, a device and molecular biological methods are disclosed for integrated nucleic acid (DNA, RNA, cDNA, etc) extraction and fractionation of different molecular weight nucleic acid molecules, from biological and clinical samples for downstream applications such as, but not limited too, polymerase chain reaction (PCR), Helicase-dependent amplification (HDA), recombinase polymerase amplification (RPA), Hybridization (such as southern blotting, microarrays, expression arrays, etc), DNA sequencing (including integrated extraction and size selection for paired-end sequencing) and other related applications.

Methodologies for analyzing the sequence and biology of DNA or RNA presently used in the art merely collect all DNA present in a biological or clinical sample. None separate based on the size, or molecular weight, of the DNA fragments, or genomes. Separation of DNA fragments based on molecular weight provides a method for enriching samples for specific DNA of interest. For example, a molecular diagnostic test for a blood born bacterial infection would benefit from enriching the sample for molecular weight DNA in the size range of the bacterial genomic DNA (gDNA) and discarding smaller fragments of DNA and larger fragments of Human DNA.

Fractionation is defined as a separation process in which a certain quantity of a mixture (in this case, different molecular weight DNA fragments) is divided up in a number of smaller quantities (fractions) in which the composition changes according to a gradient (i.e. different DNA fragments are organized by molecular weight, size or charge). Fractions are collected based on differences in a specific property of the individual components (such as molecular weight). There exists no technology that is capable of both extracting and fractioning different molecular weight DNA molecules, in one single flow-through device. This invention is therefore concerned with the fabrication and use of technologies for DNA extraction and fractionation of different molecular weight fragments of DNA (such as virus', plasmids, gDNA, etc) from whole or lysed samples in a single flow-through device.

Next generation sequencing methodologies rely on a technique called paired-end, or mate-pair sequencing, to resolve structural elements. This protocol requires that template DNA molecules to be sequenced must be within a set size, or molecular weight range. Much work and resources are spent preparing these libraries used for paired-end sequencing. Using the all in one, integrated device, as presented in this application, for both extracting DNA and selecting for the DNA molecular weight, will eliminate this bottle neck and presents the opportunity to automate sample preparation by ‘front-ending’ sequencing devices with the device presented in this patent.

A method of fabricating a microfluidics device and methods of extracting DNA and fractionating it based on molecular weight, using the microfluidics device is disclosed in accordance with embodiments of the present invention. The method comprises providing a microfluidic cassette and suggested protocols and examples for the use of this cassette for nucleic acids extraction and fractionation in one single flow-through device.

The basic concept is a single flow through fluidics device that both extracts from a lysate or whole sample, and fractionates the DNA based upon fragment molecular weight. The simplest device consists of a sample entry channel or port, an extraction and fractionation chamber, channel or column and an eluate exit port or fluidics channel. The channels, chamber or columns are most usually in the micro dimensions, but can also be in the macro, nano and pico dimensions. The unique aspect of the invention is the single chamber that both extracts and fractionates the DNA from whole samples or lysates.

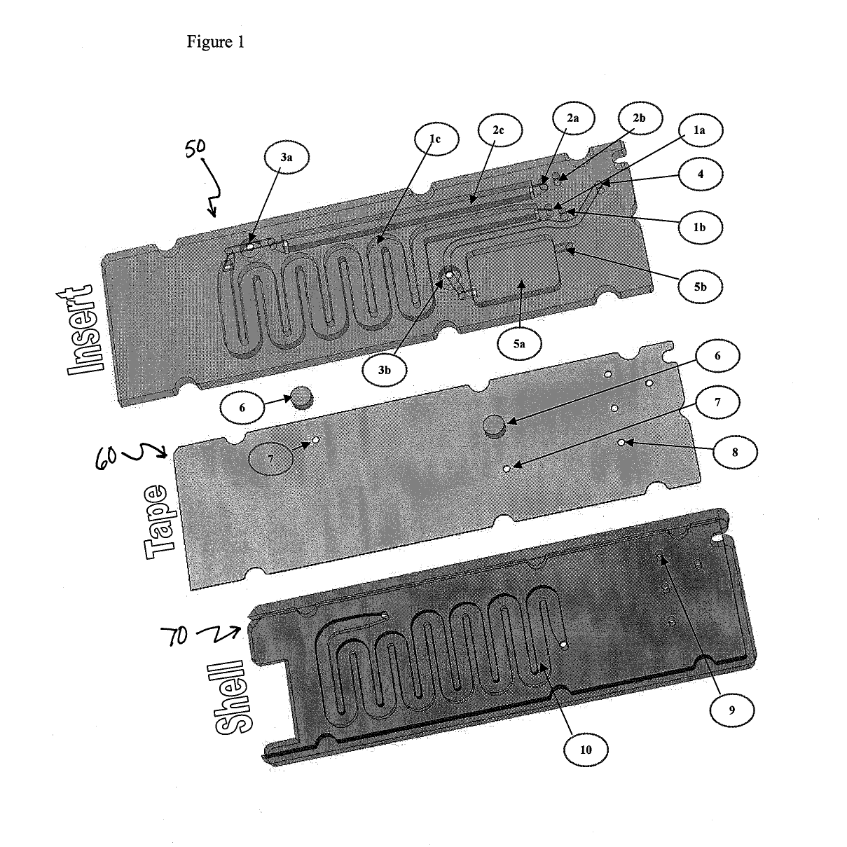

To test the possibility of DNA extraction and fractionation in a single channel/chamber a simple microfluidics device was created in accordance with embodiments of the invention. The device was fabricated and utilized as described below and with reference to FIGS. 1-12.

Blank components (shell and insert) of the cartridge are injection molded using various polymers dependant on application requirements (e.g. PP, PE, PC, COP, COC, PMMA, etc.), milled, or other manufacturing process. If necessary the channels are further milled and processed into polymer to specific depths and widths required. Cartridges are cleaned thoroughly before assembly. An adhesive is laser cut to correspond to ports required to interface with entrance and exit macro-, micro-, nano, pico-fluidics and provide flow channels between components in the cartridge Hydrophobic filters which retard the escape of sorbent material are manually punched, cut, or other method to correct size. The sorbent is vacuum filled into the sorbent chamber and sealed in with hydrophobic filters. Multiple layers are aligned and held together with Intermediate layers (such as PSA). Cartridge components are firmly bonded together using a pressure based system (e.g. lamination, hydraulic press). In preferred embodiments of the device, the cassette will be injection molded or milled from a polycarbonate, or other biocompatible plastic material. In other embodiments the cassette will be molded or milled from glass.

Some elements/features of the microfluidic nucleic acid extraction cassette are a chamber that can separate DNA from all the other cellular components and also fractionate the DNA in the sample based on molecular weight. Within the chamber are filters and/or chromatography columns that either separately or together, separates the DNA from all the other constituents within a lysate, or whole sample and also separates the DNA fragments based upon size. In many of the examples presented below and in much of the work presented to study these devices, the method of extracting the DNA is provided by a sorbent filter (such as that described in U.S. Pat. No. 7,018,538; incorporated herein in its entirety by reference), which when packed at a certain density and in an elongated channel or chamber, also causes the DNA to fractionate based upon size. The sorbent is preferably a support at least partially covered by a polymeric coating comprising polyaniline or derivatives thereof. The use of this sorbent filter at different packing densities and within the microfluidic device design, that elongates the filter, in combination, create the surprising ability to fractionate as well as extract DNA.

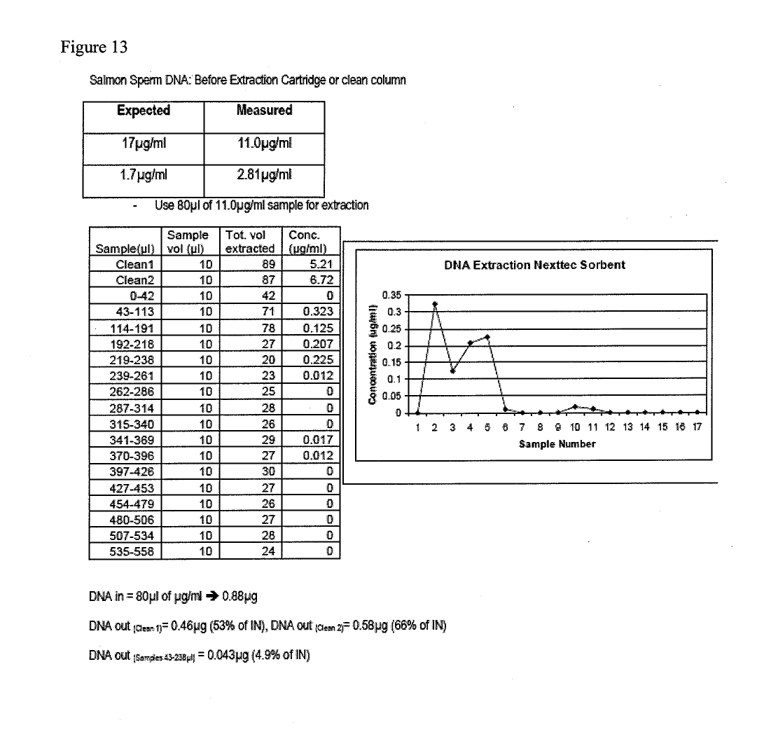

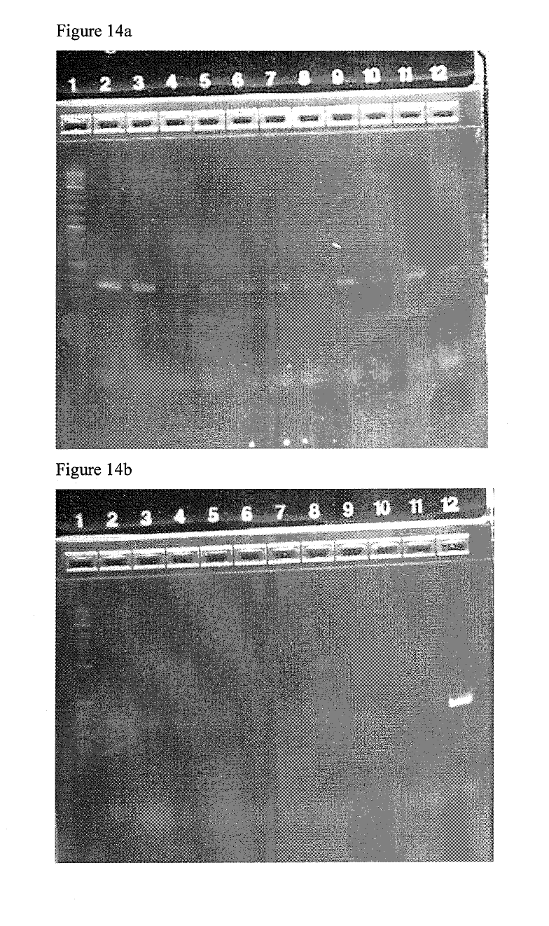

By increasing the packing density and studying the retention of different molecular weight DNA fragment populations as they passed through the channel packed with the sorbent filter, it was observed that different molecular weight DNA fragments elute at different times. FIG. 13 shows unsheared salmon sperm genomic DNA eluting in the first 5 eluate fractions and then in FIG. 14 smaller fragments from a 0.1-10 kb log ladder elute in fractions 6-10. The smaller molecular weight DNA fragments are impeded and the larger fragments pass through first. The results clearly provide evidence that both extraction and fractionation is possible within a single, or multiple channel/chamber(s) to increase resolution.

In other embodiments of the present invention, alternative filters or structures or chemistries can be used to extract and fractionate the DNA from a sample. Other filters or macro-, micro-, nano-, pico-structures capable of binding all other cellular debris from a sample lysate, or whole blood sample, can be deployed with fractionating provided by other standard chromatography columns, or a gel, or electric field, integrated with these features or chemistries.

In yet more embodiments, channel shapes, dimension and paths may be used. The example illustrated in FIGS. 1-12 show the channel to meander with micro dimensions. However the channel, or chamber dimensions are not particularly limiting and can alter, or taper, or follow other designs, as long as the channel or chamber is sufficiently long to facilitate both DNA extraction and fractionation of DNA from a lysate. The channel dimensions can be in the macro-, micro-, nano-, pico-, range, as long as filter, or chromatography material can be packed in to them.

In one embodiment, the microfluidics chamber is a bulbous structure at the entrance which tapers to a thinner exit (see e.g., FIG. 16). The bulbous end is packed with a material that facilitates spreading of the sample which is loaded through the macro-, micro-, nano-, pico-fluidic channel This then allows for the sample to be loaded onto the extraction and fractionation filter at the same time, thus allowing for better fractionation.

In some embodiments of the device, reagent reservoirs may be provided in the microfluidics device and these could provide on cartridge storage for activation buffers, sample reservoirs for eluate collection and waste.

In yet more embodiments, different pressures and flow rates are exerted and applied to the single flow-through channel to facilitate efficient molecular weight DNA extraction and fractionation. These pressures and flow rates can be altered to suit different applications, along with packing densities and channel/chamber dimensions.

One of the issues with using a high surface area in microfluidics is the high surface area to volume ratio, which in this case presents the possibility that DNA will non-specifically bind to the surface and not elute. Surface chemistries known to those skilled in the art may be employed to prevent DNA absorbing on the cassettes.

In some embodiments, the surface of the microfluidic channels may be treated such as to prevent absorption and adsorption into and onto the material. Such surface treatment may comprise of methods including but not limited to; flowing a sacrificial substance through the channel, thereby reducing loss of material, treating the surface with biological material such as bovine serum, polymerase enzymes or other such materials, or chemically treating the surface to prevent loss. Treatments may include but are not limited to the placement of materials that create a hydrophilic or hydrophobic surface to allow a smoother flow. In some other embodiment's fluorocarbons and similar materials (Teflon, as an example would act as a hydrophobic barrier, or polyacrylates) may be deposited onto the surface of the channels. Other methodologies such as UV coatings and polymer brushes that are chemically grown off the surface may also be included in this invention. In yet another embodiment it may be that the material that the cartridge is fabricated from is chosen or adapted in its design and material make-up, to prevent loss of material onto or into the surface.

With reference to FIG. 1, there is shown 1a. Buffer Filling Port—Pipette tip, or other device used to fill device with buffer, inserted into port for filling cartridge with prep buffer. 1b. Syringe Pump 3 Port—Port cap connects Buffer Port (1a) to Buffer Port (1b). This via travels the thickness of cartridge to interface external syringe pump or other device for creating flow or pressure. 1c. Buffer Reservoir—a buffer is contained or stored in this reservoir and is used to activate, or wet the filter, until point of use. 2a. Sample (Lysate) Filling Port—Pipette tip, or other device used to fill with sample/lysate, inserted into port for filling the microfluidic device with sample/lysate. 2b. Syringe Pump 1 Port—Port cap connects Sample Port (2a) to Syringe Port (2b), This via travels the thickness of cartridge to interface external syringe pump. 2c. Sample Reservoir—Sample (Lysate) contained in this reservoir, until point of use. 3a. Filter cavity 1—Holds the filter at the inlet of the sorbent-packed chamber. Alternatively, micro structures and other materials for separating DNA from a lysate may be used. 3b. Filter cavity 2—Holds the filter at the outlet of the sorbent-packed chamber. 4. Waste Channel and Port—Waste path for the excess prep buffer from out of the sorbent chamber, and off the cartridge via syringe pump 2. 5a. Collection Reservoir—Eluate from sorbent chamber collects in this reservoir for later extraction with pipette. 5b. Syringe Pump 4 Port—Provides suction to draw eluate through sorbent-packed chamber and into collection reservoir. 6. Filters—Vyon® hydrophilic filters. 7. Filter Vias—Buffer/sample lysate, or other sample passes from cartridge Insert layer to Shell layer through these vias. 8. Syringe Pump Vias (tape)—The vias at this end of the tape form channels from the cartridge Insert layer to Shell layer, where they interface with the external syringe pumps. 9. Syringe Pump Vias (shell)—Interface with the external syringe pumps. 10. Sorbent-Packed Chamber, which contains a sorbent powder, e.g., a Nexttec sorbent powder.

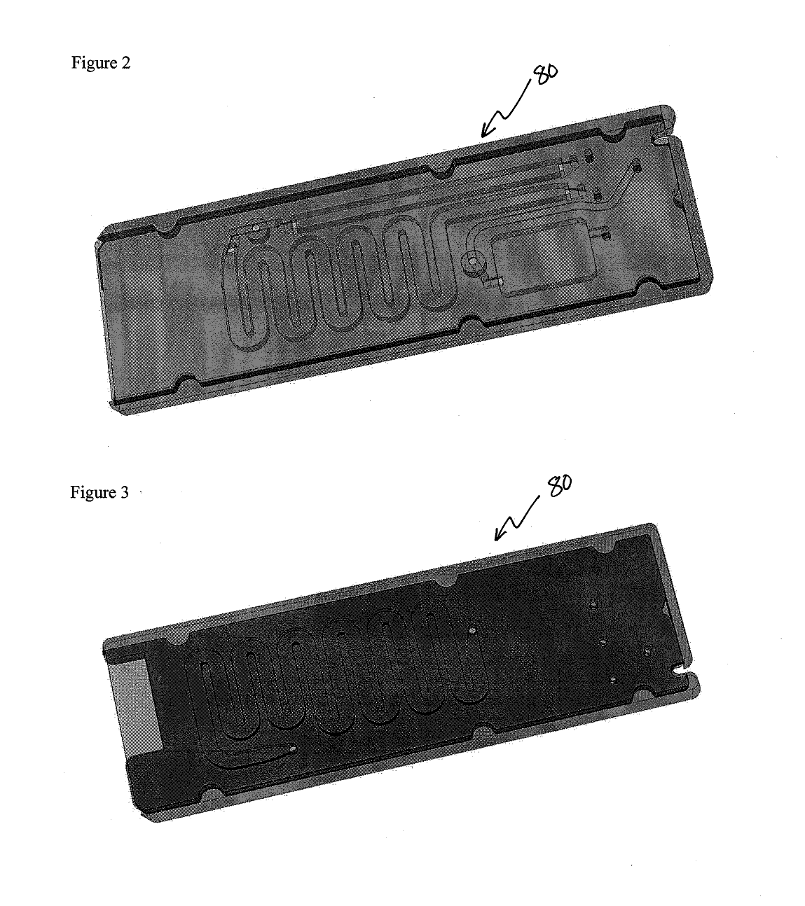

With reference to FIG. 2, the top view of an assembled microfluidic device is shown. With reference to FIG. 3, the bottom view of an assembled microfluidic device is shown.

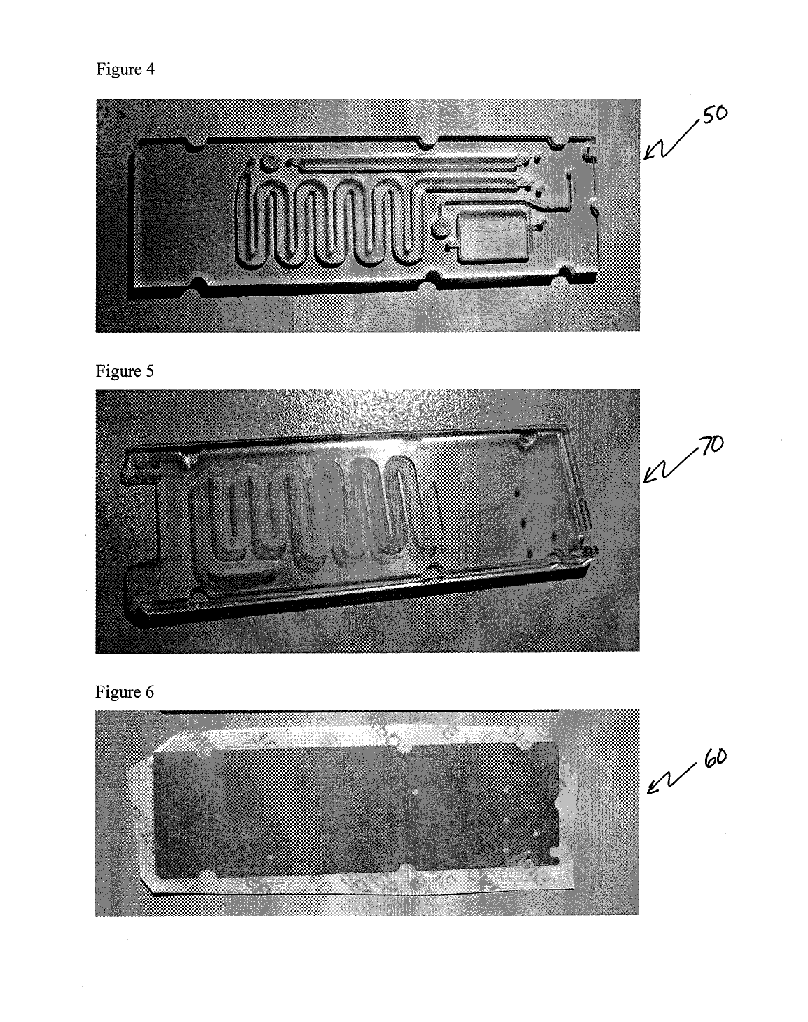

The insert of a microfluidic device shown in FIG. 4 may be created by milling, lithographically made, or other manufacturing process. It may be made of a plastic, such as polycarbonate, or other material.

The shell shown in FIG. 5 may be created by milling, lithographically made, or other manufacturing process. It may be made of a plastic, such as polycarbonate, or other material.

With reference to FIG. 6, a laser-cut double-sided tape layer is shown. The double-sided tape 60, or any other adhesive known in the art, may be used to bond the insert (FIG. 4; reference No. 50) and shell (FIG. 5; reference No. 70) together, thereby forming the microfluidic channels of the microfluidic device 80.

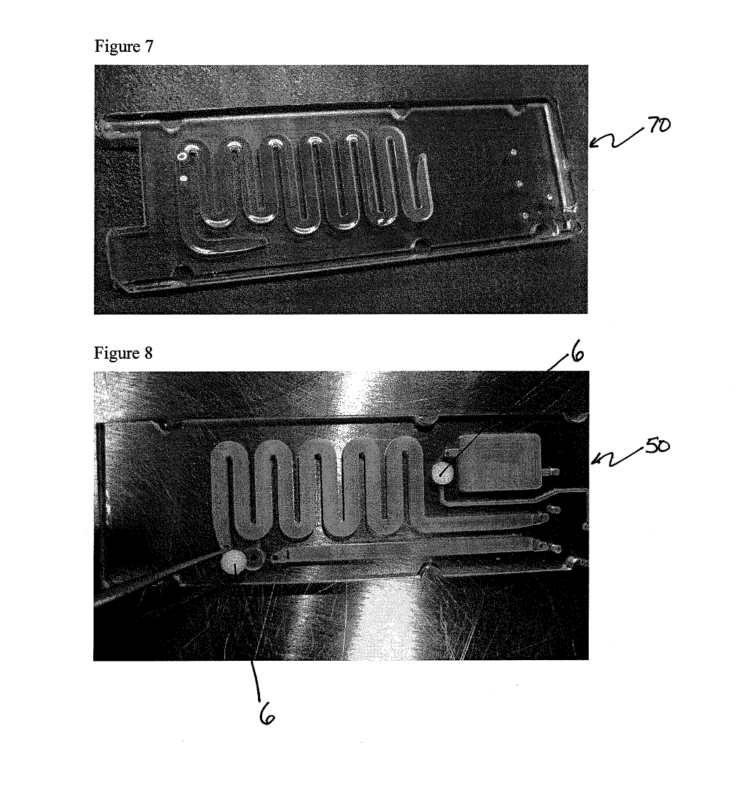

With reference to FIG. 7, the shell 70 is shown laminated to cap fluid reservoirs. Other means for capping fluid reservoirs may be employed in other embodiments.

With reference to FIG. 8, filters 6 have been placed into the filter cavities (3a and 3b) of the insert 50 at the inlet and outlet to the sorbent-packed chamber to ensure that the sorbent material remains in place. In other embodiments, these filters may not be used, as the cassette design (the assembled insert and shell components) may be structurally modified to prevent the sorbent material from escaping, e.g., by decreasing the cross-sectional luminal area of the sorbent-filled chamber at the inlet and outlet.

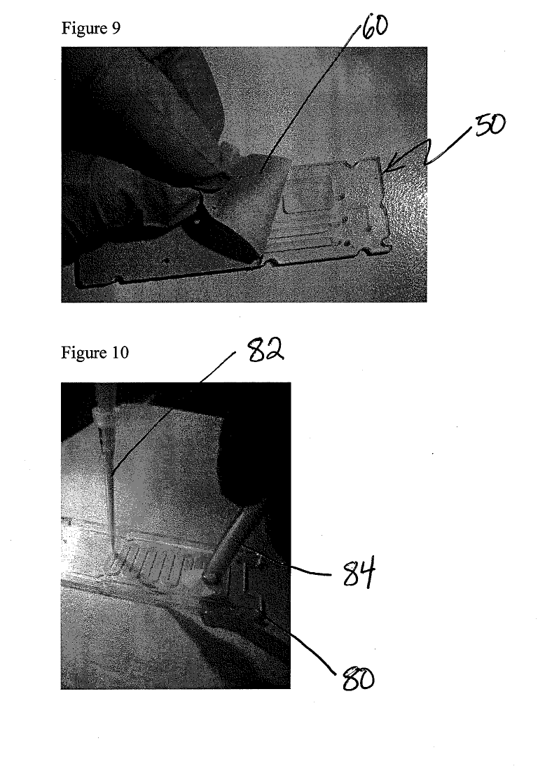

With reference to FIG. 9, a double-sided tape layer is shown being applied to the insert to hold the cassette halves (insert and shell) together and to create the microfluidics channels. Of course, any other art-recognized bonding tapes, adhesives, polymer layers, etc., may be employed instead of the double-sided adhesive tape.

With reference to FIG. 10, the sorbent chamber of the assembled cassette 80 is illustrated being filled under vacuum. The sorbent is supplied at one end of the sorbent chamber by an applicator 82, e.g., a pipette. Sorbent filling is facilitated in the illustrated embodiment by applying a vacuum to the other end of the sorbent chamber. The vacuum is applied by a vacuum tubing 84 (connected to a vacuum source). The distal end of the vacuum tubing 84 may be modified as illustrated by including or interfacing with an elastomeric suction cup to provide adequate application of negative pressure to the opening to the sorbent chamber.

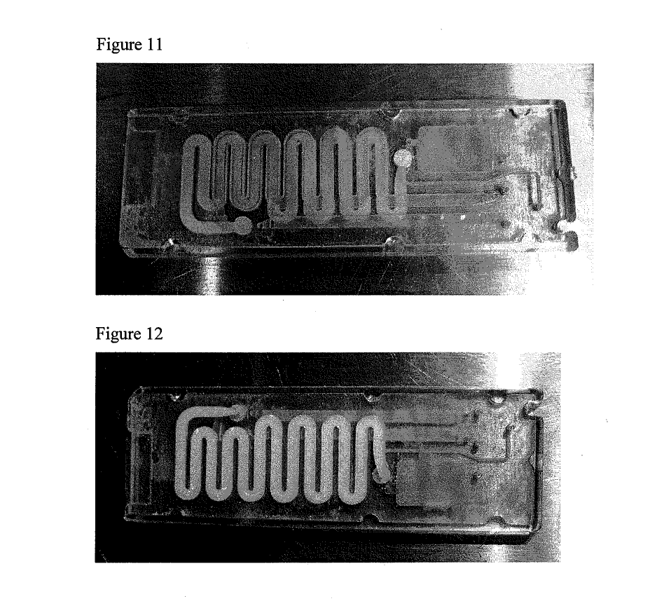

With reference to FIG. 11, the top view of an assembled nucleic acid extraction microfluidics device is shown.

With reference to FIG. 12, the bottom view of an assembled nucleic acid extraction microfluidics device is shown.

With reference to FIG. 13, the results from running 80 μl of 11.0 μg/ml salmon sperm through an extraction experiment using the extraction cassette and bench marking it with Nexttec clean column DNA extraction is presented. As the eluate came off the cassette each fraction was assessed for DNA concentration using the Quant IT system. The results from the extraction from the clean columns and each eluate fraction from the cassette are tabulated and the fraction concentrations illustrated graphically next to this. The final total DNA extracted 0.043 ug, compared to 0.46 ug & 0.58 ug on the clean columns. These results demonstrate that DNA was both extracted and fractionated using the microfluidic cassette. While the amount of DNA and the concentration was significantly lower from the extraction cassette than the clean columns, the extraction cassette was not integrated with a thermal cycler to perform a single amplification reaction. Amplification would be expected to substantially increase the DNA yield.

With reference to FIG. 14, PCR from eluate fractions from lysed human blood passed through the extraction microfluidics device was performed. FIG. 14a shows the mass ladder in lane 1 and eluates 1 through 11 in lands 2 to 12. The strongest intensity bands are in the first two fractions as expected, as these will contain the gDNA. FIG. 14b shows the mass ladder in lane 1 with lanes 2-10 containing eluate fractions 13 to 21. Lane 11 contains a PCR from water (the blank) and lane 12 a PCR from an extracted DNA sample with a known concentration of DNA (21 μl/mL).

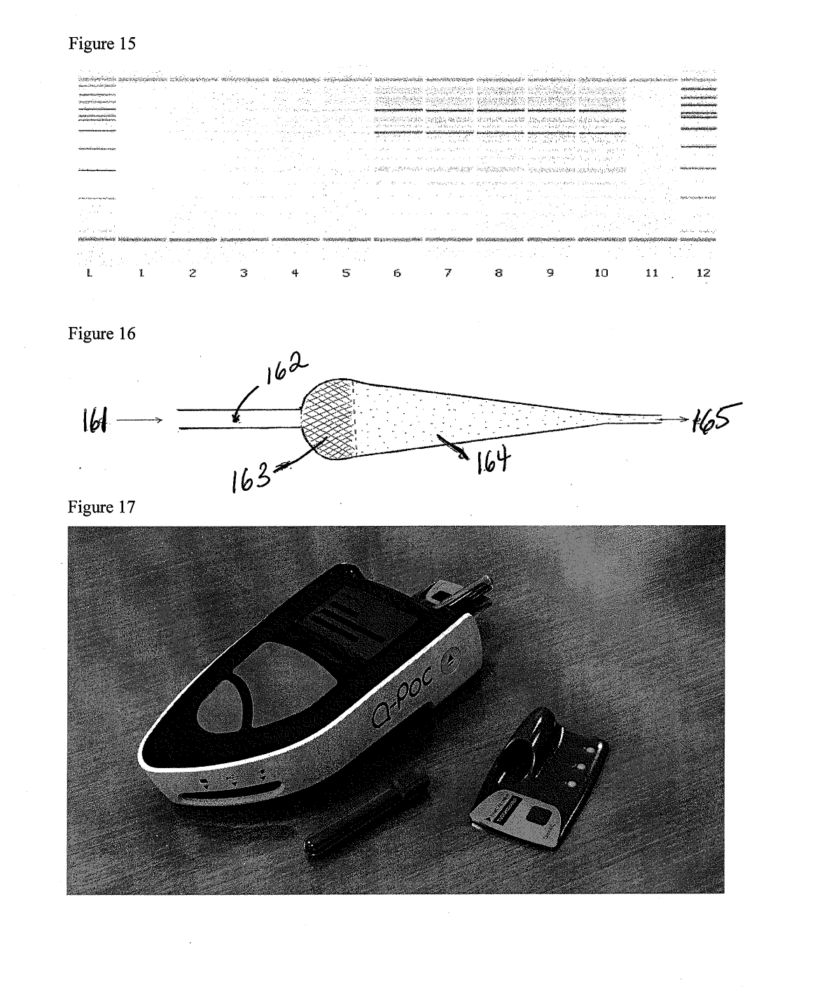

With reference to FIG. 15, a gel image from a BioAnalyzer analysis of eluate fractions that separate DNA based upon size is illustrated. The size ladder included in the BioAnalyzer kit, are run in lanes L and 12. Lanes 1-10 represent the first 10 μl eluate fractions to come off the microfluidics device presented in FIGS. 1 to 12, after 20 μl of ‘Quick-Load 2-log DNA Ladder’ (#N0469S, New England BioLabs) was loaded and run through the device.

With reference to FIG. 16, a non-limiting design of an alternative microfluidics design for the structure of a DNA extraction and fractionating device is shown. The sample entry (161) is shown. The fluidic channel (162) may be a macro-, micro-, nano-, or pico-scale fluidic channel Sorbent material (163) is depicted, which facilitates spreading of the sample which is loaded through the fluidic channel, such that the sample is loaded on to the filter (164). Preferably, the sorbent material (163) does not bind or fractionate the DNA, although it can be designed to bind and impede certain other lysate constituents. The filter (164) both separates DNA from other lysate constituents and fractionates the DNA based on molecular weight. The chamber/channel is tapered and the packing density of the filter is such that the fractionation increases the resolution of the fractionation. A macro-, micro-, nano-, pico-scaled fluidic channel (165) is where the eluate fractions run off.

FIG. 17 is a perspective view showing a point of care device and microfluidics cassette that includes the DNA extraction and fractionation device within the cassette.

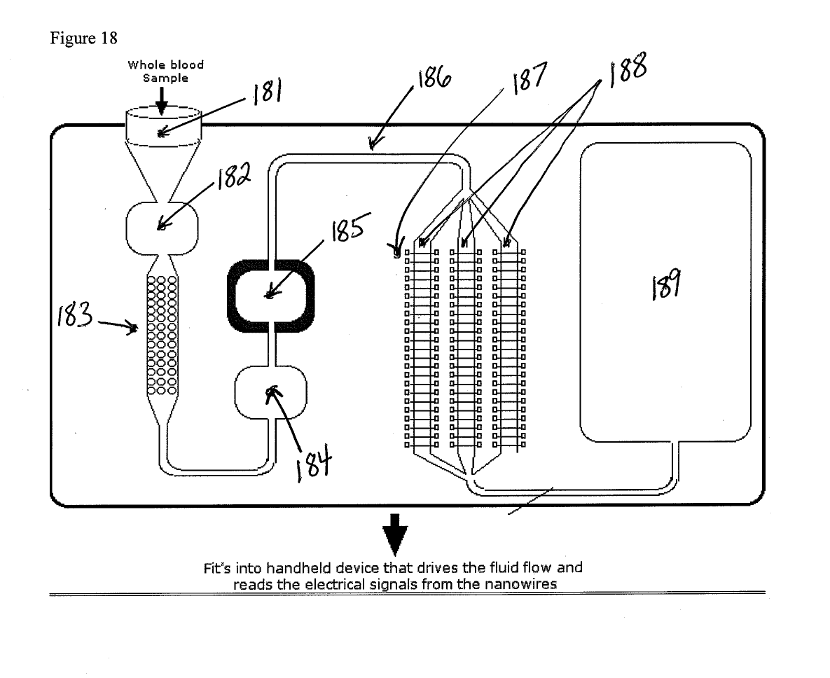

FIG. 18 is a schematic view showing the components of a microfluidics cassette designed for handheld diagnostics in accordance with some embodiments. The disposable cassette includes a sample reception 181 area and a sample lysis chamber 182. The specific lysis buffer/conditions are known in the art. Sample preparation occurs in the microfluidic channel 183, preferably with a sorbent, e.g., Nexttec's sorbent material. Concentration of the DNA may in some embodiments occur in chamber 184 following DNA extraction. Reconstitution of lyophilized reagents (if using dry reagents) and/or PCR reagents may also occur in chamber 184 (or mixing of wet reagents). Thermal cycling if employed, e.g., for PCT amplification, would occur in chamber 185. The processed (and optionally amplified) DNA would travel through microfluidic channel 186 to analytic/sensor arrays 187, comprising e.g., nanowires or other biosensors arrayed after or within the present invention. Electronics 188 are used to link the signal from the nanowires/biosensors to the reader device (not illustrated). Waste is simply an empty microfluidics reservoir 189.

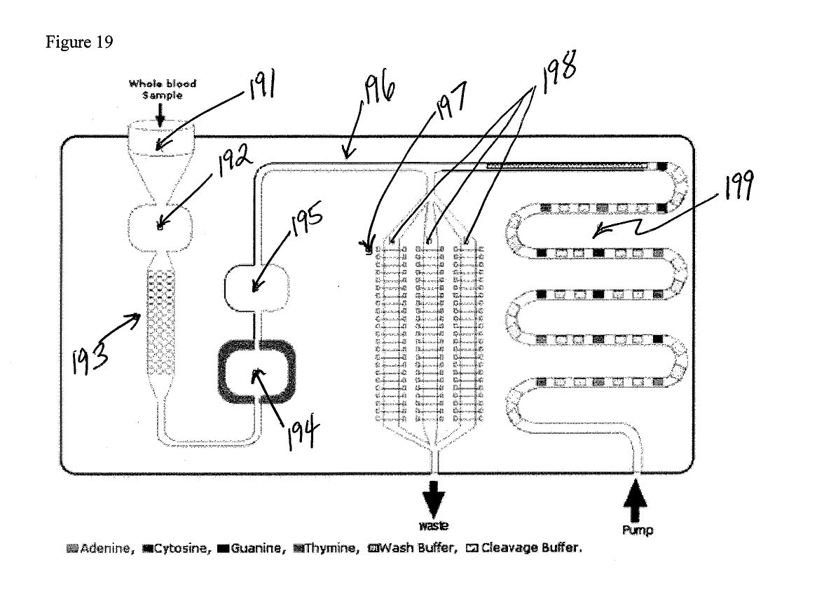

FIG. 19 illustrates a microfluidics cassette designed for handheld sequencing in some embodiments. Sample reception area 191 may act as a barrier for the sample to escape and can yet be able to accept samples, for example, much like the rubber top on blood vacutainers. The lysis chamber 192 may be a simple microreactor chamber, which comprises a lysis reagent to break up the cells and to release genomic DNA. This section might also resemble a filter to remove blood cells if the target nucleotide polymer can be free in the blood serum. A nucleic acid sample preparation chamber 193 may be used to isolate and extract the nucleotide polymer fraction of the sample from the rest of the sample constituents (proteins, carbohydrates, lipids, etc). This can be achieved by some methods well known to those skilled in the art. For instance, this maco-fluidic chamber might contain Nexttec's filter technology. Amplification of the target nucleotide polymer may occur in a cycler 194 configured for PCR amplification of the target nucleotide polymer. The cycler 194 may employ heating elements or other well known strategies of cycling a reaction mix through the different temperatures required for PCR, to perform the thermal cycling required, or isothermal amplification methods (such as LAMB, RPA, etc), which may not require heating of the sample. Sample processing, if employed, may be desired at least in some embodiments to concentrate the nucleic acids, or remove ‘over-hang’ nucleotide chains that might cause background signal, prior to sequencing. Such processing would take place in chamber 195. General microfluidics 196 includes variables such as the size of the channels, fluid flow, valves and control, materials and valves used in some embodiments. Metal connectors 197 connect the sensitive detection nanostructures (in preferred embodiments, nanowires) to the detector device (not shown) in some embodiments. Sensitive detection nanostructure arrays 198 may contact the microfluidics channel(s) and can be tightly arrayed sensitive detection nanostructures (such as nanowires, or carbon nanotubes). Methods of positioning DNA in the channel may include e.g., tight channels that allow long stretches of DNA to uncoil, migrate & stretch down the channels which may allow for long read lengths if necessary, and tiling probe/primers can be spotted on to nanowire clusters and short multiple parallel sequencing reactions performed throughout the channels. A weaving microfluidic channel 199 can be filled with reagents, in some embodiments separated by air bubbles. As this microfluidics channel can be pumped, or a tiny actuator moves the reagents along, the sequence of the reagents in the microfluidics channel can run the sequencing by synthesis reaction as disclosed e.g., in 2011-0165572 A1, 2011-0165563 A1 and PCT/IB2009/005008; incorporated in their entireties herein by reference thereto. Alternatively, this method of reagent storage can be replaced with reservoirs, or blister packs and also lyophilized reagents that are reconstituted by the reaction solution itself.

### EXAMPLES

The followings are some illustrative and non-limiting examples of some embodiments of the present disclosure.

### Example 1

### DNA Extraction from Mechanically Lysed Whole Blood, for PCR

Whole human blood was mechanically lysed by forcing it through small diameter pores, which caused the cell walls to disrupt and release the DNA (Other mechanical lysis methods can also be used). This lysate was passed through the DNA extraction microfluidic device and eluate fractions collected. These eluate fractions where used as templates in a PCR reactions that amplified a short region on the human genome. The first two eluted fractions were 34 μl and 32 μl respectively, with the next 18 fractions with 20 μl volumes. The data shown in FIG. 14 clearly demonstrates that the device is capable of extracting DNA that doesn't contain impurities that deleteriously affect polymerase action.

### Example 2

### DNA Fragment Fractionation

40 μL of 2× log ladder was loaded on to the example cassette (as described in FIG. 1-12), and pumped into the DNA extraction and fractionation column 10 μL fractions where then collected for a total of 400 μL. Each eluate fraction was then analyzed for size distribution and abundance on a BioAnalyzer device (Agilent). FIG. 15 shows the results for this. A control experiment was carried out with the sample protocol with unsheared salmon sperm DNA passed through a DNA and Extraction cassette (as described in FIG. 1-12). The results are shown in FIG. 13 and show the larger salmon sperm eluting from the device earlier than the smaller log ladder, demonstrating simple fractionation ability.

### Example 3

### DNA Sequencing

Many sequencing methodologies, helicos, FLX (Roche), Genome Sequencer (Illumina), Nanopore and nanowire (QuantuMDx, US61 094,006), utilize microfluidics and flow cells to deliver a sample of extracted DNA to where the sequencing reaction is performed. However, all of the technologies to date employ ‘off-device’ sample preparation, wherein the sample has it's DNA (or RNA/cDNA) extracted using traditional bench top methodologies (Qiagen's spin columns, Invitrogen's Charge Switch, Nexttec's sorbent filter spin columns, etc) which adds significant time to the sequencing process and demands significant operator hands on time. This microfluidics solution will ‘front-end’ these technologies, allowing for each technology to become a sample to result device and automate all of the processed required to generate sequence data.

### Example 4

### Automatic and Integrated DNA Extraction and Size Selection for Paired-End Sequencing

Standard next generation sequencing, using the shotgun method is limited by short read lengths. A solution to this critical limitation is the paired-end tag (PET) sequencing strategy, in which short and paired tags are extracted from the ends of long DNA fragments for ultra-high-throughput sequencing. The PET sequences can be accurately mapped to the reference genome, thus demarcating the genomic boundaries of PET-represented DNA fragments and revealing the identities of the target DNA elements. To do this extracting DNA is followed by selection of specific sized fragments to allow for the successful implementation of this strategy. This is time consuming and expensive.

For instance, the Illumina HiSeq and MiSeq protocol includes one Zymo cleanup step (following “tagmentation”) and one Ampure XP size selection step (following limited cycle PCR). The final library have a median insert size of ˜250-300 to support long paired end 2×150 read lengths on the MiSeq system.

Using the all in one, integrated device for both extracting DNA and selecting for the size will cut this sample prep bottle neck down and presents the opportunity to automate it by front-ending sequencing devices with the device presented in this patent.

### Example 4

### Diagnostics

Many molecular diagnostic methodologies, Cepheid (Smart Cycler), Light Cycler (Roche), BeadXpress & Eco Real-Time PCR system (Illumina), 7500 Real-Time PCR System (ABI), GeneChip system (Affymetrix), and future devices such as Nanopore device, microfluidics devices and nanowire & carbon nanotube devices (QuantuMDx), utilize DNA (or RNA/cDNA) as their test substrate. However, most, if not all of the technologies to date employ ‘off-device’ sample preparation, wherein the sample has it's DNA (or RNA/cDNA) extracted using traditional bench top methodologies (Qiagen's spin columns, Invitrogen's Charge Switch, Nexttec's sorbent filter spin columns, etc) which adds significant time to the molecular diagnostic process and demands significant operator hands on time. This microfluidics solution will ‘front-end’ these technologies, allowing for each technology to become a sample to result device and automate all of the processed required to generate molecular diagnostic data.

### Example 5

### Single Device Molecular Analysis

In some embodiment, specific target DNA sequences may be extracted in a microfluidics channel that leads to further downstream processes in a single flow-through microfluidics cassette. The cassette will perform Lysis, extraction, sample concentration, amplification, detection and waste handling, or any combination of these processes. The cassettes may be fully enclosed, disposable and be operated by a handheld, or benchtop, or high throughput device.

### Example 6

### Handheld Sequencing Device

In some embodiment, specific target DNA sequences may be extracted in a microfluidics channel that leads to further downstream processes in a single flow-through microfluidics cassette wherein the DNA is sequenced (FIG. 13). All the reagents required for the lysis and extraction of DNA from samples can be stored in a microfluidics channel, each wash solution and lysis buffer, can be separated by an air bubble, or another method of separating the reagents, or the reagents can be stored in blister packs, lyophilized or other methods for storing reagents within microchannels.

In some embodiment, small specific regions of target viral, bacterial or genomic DNA can be extracted for sequencing and therefore be diagnostic for the presence or absence of a specific virus, bacteria, or genetic sequence (such as a SNP), as well as provide value-added information on genetic type, mutations (known or unknown), drug resistance status, etc.

Various embodiments used in connection with the present disclosure lend itself to handheld sequencing as it may not require bulky and equipment required to extract the DNA prior to the sequencing reaction.

In one embodiment, a probe sequence can be immobilized on a sensitive detection nanostructure (in this case a nanowire) and the template ssDNA molecule to be sequenced can hybridize to the probe sequence and the probe sequence can act as a primer for the sequencing by synthesis reaction. In another embodiment the template ssDNA molecule can be immobilized to the sensitive detection nanostructure and can be primed for sequencing with a free primer oligonucleotide.

### Example 7

### Design of a High Resolution Microfluidic DNA Extraction & Fractionation Device

One of the problems with fractionating a sample by introducing it to a column from a microfluidics channel of similar density is that not all the sample is phased at the same time. A mixture of DNA fragments of different molecular weight fragments will most probably be homogeneously spread in the sample, throughout the microfluidics channel and thus fractionation will be hampered by the sample entering the extraction and fractionation filter over a period of time with the first DNA to be loaded running out of phase of DNA molecules that enter last. Therefore the mixture needs to be phased into the extraction and fractionation column at the same time. A microfluidic chamber consisting of a bulbous entrance that is filled with a material that facilitates the spreading of the sample without affecting the fractioning of the sample, which therefore allows for the whole sample to enter the fractionation column at the same time, immediately followed by a tapering (in terms of chamber dimensions) column, packed with a material that both separates the DNA from the lysate constituents and fractionates the DNA based on fragment molecular weight, is a solution. A non-limiting design of such a possible microfluidics design for the structure of a DNA extraction and fractionating device is presented in FIG. 16.

What is claimed is:

1. A device for simultaneously extracting and fractionating DNA from a lysate or a whole sample, the device comprising a single flow-through microfluidic channel, the channel comprising buffer and reagent chambers and a sorbent filter.

2. The device of claim 1, wherein the sorbent filter comprises a support at least partially covered by a polymeric coating comprising polyaniline or derivatives thereof.

3. The device of claim 1, wherein the device comprises a glass material.

4. The device of claim 1, wherein the device comprises a PDMS material.

5. The device of claim 1, wherein the single flow-through microfluidic channel comprises a bulbous structure at an inlet thereof and wherein the bulbous structure tapers to a thinner structure at an outlet thereof.

6. The device of claim 1, wherein the sorbent filter comprises a series of solid or hollow microstructures fabricated into the walls of the microfluidc channel.

7. The device of claim 1, wherein the sorbent filter is lose and packed within the microfluidic channel.

8. The device of claim 1, wherein the sorbent filter is a matrix configured to bind cellular and other clinical/biological sample material other than nucleic acids.

9. A method of fabricating a microfluidics device to extract and fractionate DNA from a sample, comprising:
forming at least two blank layers having a channel;
forming an adhesive layer with ports cut to through the layer corresponding to an inlet and an outlet port of the channel;
vacuum packing a portion of the channel with a sorbent material; and
aligning the blank layers with the adhesive layer therebetween and bonding the layers together under pressure.

10. A method for simultaneously extracting and fractionating DNA from a lysate or a whole sample, the method comprising:
providing the device of claim 1;
applying the sample to an inlet of the single flow-through microfluidic channel;
activating the sorbent filter with a buffer;
flowing the sample into the portion of the channel containing the sorbent filter;
flowing the sample through the channel and looping it though the portion of the channel containing the sorbent filter from between one and ten times; and
flowing the extracted and fractionated DNA out of the device.

11. The method of claim 10, wherein the sample is flowed into the portion of the channel containing the sorbent filter and then incubated therein for between 15 seconds and 15 minutes, before being flowed out the device.

12. The method of claim 10, wherein the sample is flowed into the portion of the channel containing the sorbent filter and then oscillated back and forth within the sorbent filter channel, before flowing the sample out of the device.
